(19) United States
(12) Patent Application Publication
(10) Pub. No.: US 2022/0133193 A1
(43) Pub. Date: May 5, 2022

(54) A DEVICE AND A METHOD TO IDENTIFY PERSONS AT RISK FOR DEPRESSIVE RELAPSE

(71) Applicant: Emotra AB, Mölndal (SE)

(72) Inventor: Lars-Håkan THORELL, Linköping (SE)

(73) Assignee: Emotra AB, Mölndal (SE)

(21) Appl. No.: 17/435,304

(22) PCT Filed: Feb. 24, 2020
(86) PCT No.: PCT/SE2020/000007

§ 371 Date: Aug. 31, 2021

(30) Foreign Application Priority Data
Mar. 1, 2019 (SE) 1900039-7

(51) Int. Cl. A61B 5/16; A61B 5/00; A61B 5/0533

(52) U.S. Cl. CPC A61B 5/16; A61B 5/0533; A61B 5/7264

(57) ABSTRACT

The invention relates to a device and a method to identify a depressed person at risk for a depressive relapse or recurrence. The device measures biological signals from the brain of a person in order to detect depressed persons who are at risk for depressive relapse or recurrence. The device comprises a measuring unit that measures the electrodermal activity in the fingers of the person in order to detect depressed persons who are at risk for depressive relapse or recurrence. The measuring unit is arranged to transmit a certain experimentally well defined pattern of sound or tone signals to the person and to provide a signal for the analysis of the electrodermal response from the person in question. The invention also relates to a device and a method to perform the analysis of the electrodermal response by means of machine learning technique.

A device and a method to identify persons at risk for depressive relapse.

The present invention relates to a device and a method to identify a depressed person at risk for a depressive relapse or recurrence. The device measures biological signals from the brain of a person in order to detect depressed persons who are at risk for depressive relapse or recurrence. The device comprises a measuring unit that measures the electrodermal activity in the fingers of the person in order to detect depressed persons who are at risk for depressive relapse or recurrence. The measuring unit is arranged to transmit a certain experimentally well defined pattern of sound or tone signal to the person and to provide a signal for the analysis of the electrodermal response from the person in question.

Approximately ten percent of the world's overall population is affected by major depression, so-called Major Depressive Disorder (MDD). Depression usually strikes more than once in a lifetime and for many people it can become a chronic illness with several relapses or recurrences. Relapse is usually classified as another episode of depression that happens fewer than six months after treatment of an acute depression. A recurrence is a new episode that comes after six months or longer since the previous episode has been resolved. A depression relapse or recurrence means a new treatment of the patient, sometimes through a combination of therapies such as both antidepressant treatment and psychotherapy.

An object of the present invention is to identify those patients that are at risk for such a depressive relapse or recurrence.

The method that has been available so far in traditional health care to identify whether there is risk for relapse or recurrence has been based on personal interviews by a psychiatrist or other available health care professional and the patients age, sex, social background or the like. Such methods are characterized by being fairly subjective. The outcome of such investigations depends on the experience of the health care professional and the way of valuing the patients and therefore tend to be rather arbitrary. If a more objective evaluation of risk for relapse or recurrence of depression was possible, the higher risk patients could be offered additional care, for example more frequent medical check-ups, longer pharmacological treatment or extended psychotherapy as well as a more careful monitoring of the phasing out of the treatment.

Based on the research developed by associate professor Lars-Håkan Thorell for 25 years, the company Emotra AB, since its start 2001, has developed a clinically useful and entirely objective method that can be used as a support in the clinical evaluation of suicide risk among depressed patients. The method is a so-called EDR test (Electro Dermal Reactivity), in which the ability of the skin (derma) to lead a weak current is utilized, and which has led to the so-called EDOR test (Electro Dermal Orienting Reactivity). The measurements performed according to the EDOR test include among other things electrodermal effects based on the ability of the skin to lead a weak current via the fingertips. The more a person is responding with attention in his brain on a signal, the more sweat glands are activated. The channels of the sweat glands are filled to the skin surface, thus forming more current paths through the skin which in itself has a high electrical resistance so that a larger current is measured.

By testing the response of the patients to certain experimentally well defined pattern of sound signals or tones, it has been possible to identify those patients who are so-called electrodermal hyporeactive, i.e. persons who do not show interest in the tones. By definition, hyporeactive persons react very little on signals. This is rare among healthy individuals or depressed persons who are not suicidal. However, to react very little to the signals is very common for suicidal depressed patients. A test of this kind will take approximately 15 minutes to complete.

At the EDOR test a special apparatus is used to measure the biological signals from the brain to detect depressed patients who are at risk for suicide. The apparatus comprises a handheld measuring unit with sensor means for measuring electrodermal activity in the fingers of the patients. In addition to the handheld measuring unit the apparatus also comprises headphones and a computer for the analysis of the measurements. The measuring unit transmits an experimentally well defined, specific tone to the patient through the headphones. The tone is repeated according to a specific, tested schedule in widely varying intervals around about 40 seconds. The electrodermal activity of the patient is measured throughout the entire test which takes 15 minutes.

The measuring unit has a size similar to a conventional spectacle case. The measuring unit is placed on the desk in front of the patient/test person. Sensor means in the form of skin conductance electrodes for measuring the electrodermal activity are mounted on the upper side of the measuring unit and on which the patients are allowed to place their fingers during the test.

A device of this type has previously been described in WO 2015/047147 A1. The device described in this publication is for use specifically in the evaluation of suicide risk. It is not indicated anywhere that the device could be used for identification of a depressed person at risk for a depressive relapse or recurrence.

In said device those patients who are so-called electrodermal hyporeactive, i.e. persons who do not show interest in the tones are identified. As mentioned, hyporeactive persons react very little on signals which is rare among healthy individuals or depressed persons who are not suicidal. By experimental studies we have found that hyporeactivity, i.e. to react very little to the signals, is very common also for depressed patients at the risk for relapse or recurrence.

According to the invention the measured electrodermal hyporeactivity of a depressed person is used as a basis to identify if the person is at risk of depressive relapse or recurrence.

According to a preferred embodiment of the invention a pattern of sound or tone signal is provided by the measuring unit to the test person via headphones. The sound signal or tone is repeated according to a specific, tested schedule in widely varying intervals.

In the following the invention will be described more in detail with reference to the accompanying drawings in which,

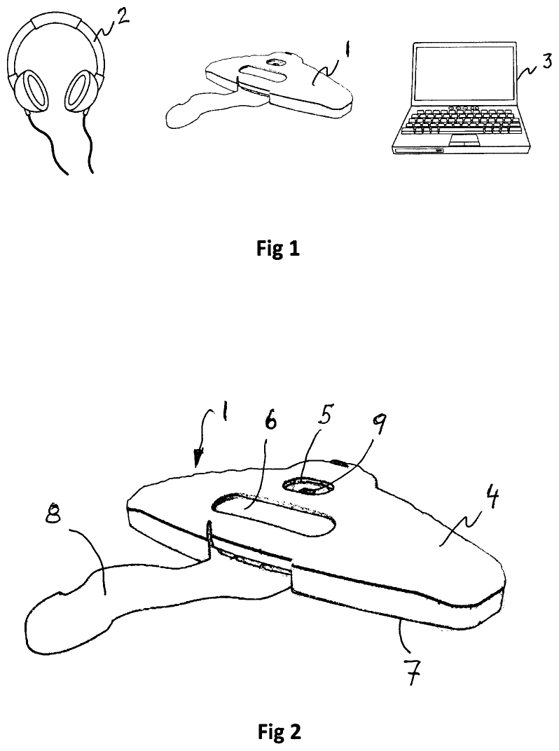

FIG. 1 schematically illustrates the main components of an apparatus for EDOR test, in which the electrodermal response from the test person is analyzed by using a laptop computer,

FIG. 2 shows a hand held measuring unit for the EDOR test,

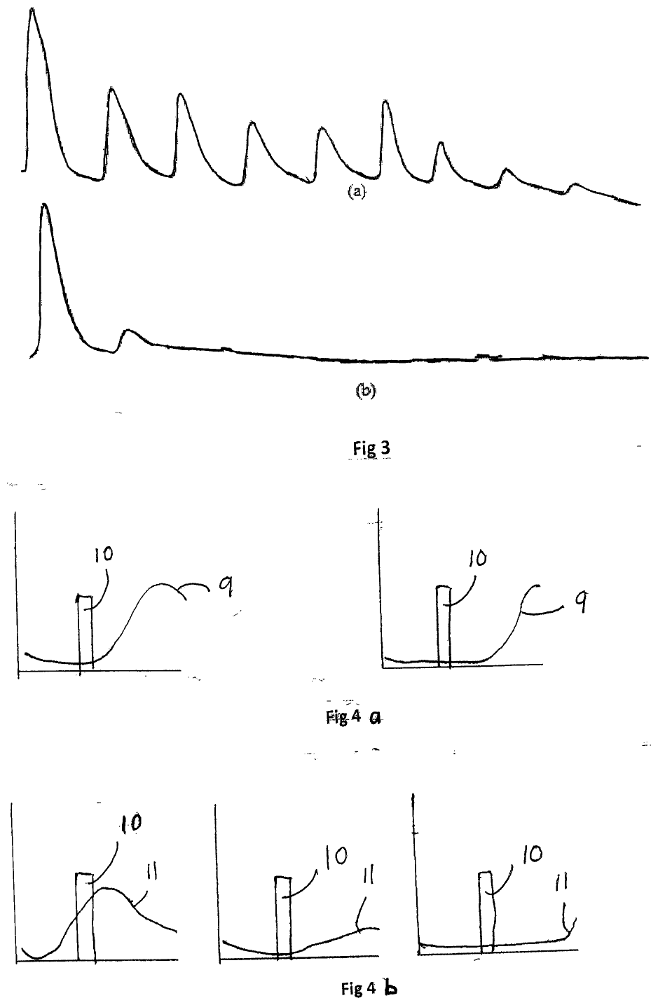

FIG. 3 shows two typical examples of electrodermal response (reaction curves) to tone stimuli from a reactive and hyporeactive person, respectively, at an EDOR test, and

FIG. 4 illustrates two examples of correct (true) reaction curves (FIG. 4a) and three examples of incorrect reaction curves (FIG. 4b) when using machine learning technique for analysing the electrodermal response.

FIG. 1 schematically illustrates the main components of an apparatus for EDOR test of the type which has been described in the introductory portion of our specification. The apparatus thus comprises an easily managed hand held measuring unit 1, a headphone 2 and a laptop computer 3 for the analysis of the electrodermal response from the measuring unit.

FIG. 2 shows an example of a hand held measuring unit 1 developed by Emotra AB. The measuring unit is designed for easy handling with an upper side 4 with two skin conductance electrodes, a smaller, circular gold electrode 5 and a larger, elongated gold electrode 6, and a bottom side 7 intended to rest against a desk or the like. The measuring unit has a size and shape substantially corresponding to a spectacle case. A measuring unit of this type is previously known and described in said WO publication WO 2015/047147 A1 and will not be described in any further detail here.

As already mentioned the EDOR test is measuring the orienting reactions from a well defined repeated event, in this case a tone. In FIG. 3 it is illustrated an example of a typical electrodermal response in the form of skin conductance from a tone stimulus for 15 minutes for a reactive as well as a hyporeactive person, curve a and b, respectively. From curve a it is evident that a reactive person learns what is usual in the environment, while the hyporeactive person ignores changes in the environment (see curve b). The hyporeactive person does not respond to the third and subsequent signals. Habituation is reached already at the third stimulus. The scale of habituation is defined as the sequence number of the first stimulus in a sequence of three that does not cause any electrodermal response. A response of this type is also previously known by said WO publication in evaluation of suicide risk of a person.

By an experimental study we have found that hyporeactivity, i. e. to react very little to the signals, is very common also for the specific depressed patients that are at risk for relapse or recurrence. So, according to our invention the above described measuring device is used for the new field of application. i. e. identification of those depressed patients that are at risk for depressive relapse or recurrence.

In the experimental study 105 patients were tested, all admitted to the hospital with the diagnosis of a depressive episode. 8 patients were excluded from the analysis because of technical test problems, interruption or the like. The equipment and software necessary to perform the EDOR test was developed by the Swedish company Emotra AB. It includes a box containing electrodes and generating the sound signal, professional earphones and a computer connected to the box by Bluetooth. During a 15-minute test, a series of sounds (1 s, 90 dB, 1 kHz) was presented to the patient through the earphones in intervals from 20 to 80 seconds in an unpredictable scheme. Changes of the skin conductance to the direct current of 0.5 V were measured and registered.

All the registered data were anonymously sent to Emotra for professional assessment by experts in EDA measurements. The possible results in the test were “reactive” or “hyporeactive”, with the criterion for the hyporeactivity being the habituation score 3 or lower (habituation score is the order number of the first stimulus in a sequence of 3 stimuli that do not evoke an electrodermal response)—meaning that there were electrodermal responses to no more than 2 stimuli within the first 5 stimuli.

One year after the EDOR test each patient was contacted and the information on their possible depressive episodes in the follow-up period was obtained. In this study the patients were not differentiated by the time when the re-appearance of a depressive episode occurred. When the term “depression relapse or recurrence” is used, it refers to a whole group of patients, including those with “recurrence”—understood as a new depressive episode of a person who had previously experienced a depressive episode and achieved recovery, as well as patients with “relapse”—understood as return of symptoms to the full syndrome criteria for an episode during partial or full remission but before recovery.

In the tested group 20 patients were hyporeactive in EDOR test and 77 were reactive. During the follow-up period, 47 patients had at least one new depressive episode. 17 patients were hospitalised during the follow-up period because of depression. 6 patients were lost to the follow-up.

In the group of hyporeactive patients, the risk of depression relapse or recurrence was greater and the difference was statistically significant (p-value <0.05), see Table 1.

According to the constructed model, hyporeactivity was associated with a 3 times higher risk of depression relapse or recurrence compared to the reactive group, see Table 2 below.

TABLE 1
Risk of depression relapse or recurrence and reactivity in EDOR test.

| | | in general | Hyporeactive | | Reactive | | | |
|---|---|---|---|---|---|---|---|---|
| Variable | | (N = 97) | (N = 20) | | (N = 77) | | Test | P-value |
| Relapse or recurrence | No | 48.4% (N = 44) | 26.3% | (N = 5) | 54.2% | (N = 39) | Fisher | 0.0397 |
| of depression | Yes | 51.6% (N = 47) | 73.7% | (N = 14) | 45.8% | (N = 33) | | |
| Hospitalisation during | No | 81.4% (N = 79) | 60% | (N = 12) | 87% | (N = 67) | Chi- | 0.0145 |
| the follow-up period | Yes | 18.6% (N = 18) | 40% | (N = 8) | 13% | (N = 10) | squared | |

TABLE 2
Logistic regression analysis of predictor (reactivity)
for risk of depression relapse or recurrence.

| | B | | | | OR (95% | |
|---|---|---|---|---|---|---|
| | (regression | Odds | | | confidence | |
| Variable | coefficient) | ratio | SE | Wald (Z) | interval) | Pr(>\|z\|) |
| Constant | 1.03 | 2.8 | 0.521 | 1.976 | (0.0085)-(2.051) | 0.0481 |
| Reactivity | −1.197 | 0.3022 | 0.5722 | −2.091 | (−2.318)-(−0.0753) | 0.0365 |

Hospitalisation during the follow-up period was also significantly more frequent in the hyporeactive group. The group of hyporeactive patients did not significantly differ from the reactive group considering any of the clinical variables like age, sex, depth of depression or the like. These findings may indicate that hyporeactivity in EDOR test is potentially even more valuable in this group of patients, as it seems to enable patients evaluation independently of their clinical characteristics.

Results of this study suggest that electrodermal reactivity could be used as a part of routine assessment during the diagnostic and therapeutic process, helping to identify patients in higher risk of depression relapse or recurrence so that they could be offered additional care—for example more frequent medical check-ups, longer pharmacological treatment or extended psychotherapy as well as a more careful monitoring of the phasing out of the treatment. In the form used in our study, i.e. performing EDOR test by means of a measuring device according to said WO 2015/047147 A1—Emotra AB, it can be easily applied by any staff member after a short training, it does not require laboratory setting and its results can be easily interpreted even without professional knowledge of EDA mechanisms as such. What is also important, it is not invasive and minimal co-operation of the patient is needed.

As mentioned our invention relates to a new method to detect depressed persons at risk for depressive relapse or recurrence. And in the above study the tested group therefore consisted of hospitalised patients with diagnosis of depression already given at the moment of EDOR test. The results should not be automatically extrapolated to other groups of patients.

According to the method described so far biometric data as reaction curves are transferred to the Emotra cloud, analysed and reported back to the clinic. Analysis of these reaction curves is a manual process that can be done after some training.

In order to improve the analysis process a more powerful tool by using machine learning techniques has been suggested. Such a technique is illustrated in the Master's Thesis by Arnaud Moulis:

Machine Learning for Suicide Risk Assessment on Depressed Patients. The use of Electro Dermal Orienting Reactivity to Identify Hyporeactive Patients. Department of Computer Science and Engineering, Chalmers University of Technology, Sweden 2019.

In these thesis it is concluded that reactivity and hyporeactivity for suicidal patients can be detected with machine learning accurately. Hyporeactive patients could be identified for the purpose of earlier treatment by automating a complex manual analysis and thereby saving valuable time of experts. It is reported that a precision of 82% and an accuracy of 89% compared to the manual process has been reached by the machine learning techniques. However, an accuracy of 89% might be insufficient for a reliable process and there is a need to improve the technique even further.

It has been found that the main reason for the discrepancy between the manual analysis and the machine learning technique is the fact that the machine learning technique does not distinguish between a true reaction (electrodermal response to a stimuli) and artefacts, distorsions in the curve and spontaneous reactions (not connected to the tone stimuli). Typical artefacts in this case could be a door slam, sneezing or the like. This might lead to a mis-interpretation in the machine learning technique so that a number of hyporeactive patients (that in fact has ceased to react on stimuli) instead are classified as reactive patients.

In order to solve this problem the machine learning technique of the present invention is arranged to react only if specific conditions with respect to the reaction curves are fulfilled.

According to the invention the conductance reactions are only considered if the following conditions are fulfilled: The reaction should happen within a certain time interval after a tone or sound stimuli, specifically within 0.8-4 seconds after the stimuli, and the slope or gradient of the reaction curve, measured from the beginning of the curve to the maximum of the amplitude of the curve, should be in the interval of 0.6-1.3.

These conditions are illustrated in FIGS. 4a and 4b in which the Y-axis is the skin conductance (amplitude) while the X-axis the time. In FIG. 4a there are two examples of correct, true reaction curves 9 having a slope within the required interval and within the required time interval after the tone or sound stimuli 10.

FIG. 4b illustrates three examples of reaction curves 11 due to artefacts, and in which the curves 11 are outside the required intervals, that is outside the required time interval and/or having an incorrect slope.

If the above conditions are fulfilled the machine learning technique will identify a significant higher number of the hyporeactive patients so that the precision in the machine learning technique is substantially increased.

It should be understood that the machine learning technique per se is not described in any further detail here, in that case it is referred to said Master's Thesis. Our invention relates to the specific conditions for the machine learning technique in order to improve the reliability as defined in the following claims in order to improve the reliability to identify if a person is at risk for depressive relapse or recurrence.

1. A device for measuring electrodermal activity in the fingers of a person wherein the device comprises a measuring unit arranged to transmit a certain experimentally well defined pattern of sound or tone signals to the person and to provide a signal for the analysis of the electrodermal response from the person in question, characterized in that the measuring unit is used for measuring the electrodermal hyporeactivity of a depressed person in order to identify if the person is at risk for depressive relapse or recurrence.

2. A device according to claim 1, characterized in that the experimentally well defined pattern of sound or tone signals comprises a series of sounds in intervals from 20 to 80 seconds in an unpredictable scheme.

3. A device according to claim 2, characterized in that the criterion for the hyporeactivity being the habituation score 3 or lower, wherein the habituation score is defined as the order number of the first sound stimulus in a sequence of 3 stimuli that do not evoke an electrodermal response.

4. A device according to claim 1, characterized in that the signal for the analysis of the electrodermal response is analysed by means of machine learning technique.

5. A device according to claim 4, characterised in that the machine learning technique is arranged to react only if specific conditions with respect to the reaction curves are fulfilled.

6. A device according to claim 5, characterised in that the machine learning technique is arranged to react if the reaction happens within a certain time interval after a stimuli, and the reaction curve has a predetermined slope or gradient.

7. A device according to claim 6, characterized in that said time interval is 0.8-4 seconds after the stimuli and
the slope or gradient of the reaction curve, measured from the beginning of the curve to the maximum of the amplitude of the curve, is in the interval of 0.6-1.3.

8. A method to identify persons at risk for depressive relapse or recurrence wherein a measuring unit measures the electrodermal activity in the fingers of the person in order to detect a depressed person at risk for depressive relapse or recurrence, and wherein the measuring unit is arranged to transmit a certain experimentally well defined pattern of sound or tone signals to the person in order to analyze the electrodermal reactivity of the person in question, characterized in that the electrodermal hyporeactivity of the person is used as a basis to identify if the person is at risk of depressive relapse or recurrence.

9. A method according to claim 4, characterized in that the transmitted pattern of sound or tone signals comprises a series of sounds in intervals from 20 to 80 seconds in an unpredictable scheme.

10. A method according to claim 8, characterized in that
the signal for the analysis of the electrodermal response is analysed by means of machine learning technique and that the machine learning technique is arranged to react only if the electrodermal reaction happens within a certain time interval after a stimuli, and the reaction curve has a predetermined slope or gradient.
